(19) United States
(12) Patent Application Publication
(10) Pub. No.: US 2017/0350874 A1
(43) Pub. Date: Dec. 7, 2017

(54) OPTICAL INTERROGATION AND CONTROL OF DYNAMIC BIOLOGICAL FUNCTIONS

(71) Applicants: Oxford University Innovation Limited, Oxford Oxfordshire (GB); The Research Foundation for the State University of New York, Albany, NY (US)

(72) Inventors: Emilia Entcheva, Stony Brook, NY (US); Gil Bub, Oxfordshire (GB)

(21) Appl. No.: 15/540,947

(22) PCT Filed: Dec. 30, 2015
(86) PCT No.: PCT/GB2015/054177

§ 371 Date: Jun. 29, 2017

(30) Foreign Application Priority Data
Dec. 31, 2014 (GB) 1423398.5

(51) Int. Cl. G01N 33/483; G01N 21/63; G06T 7/00

(52) U.S. Cl. CPC G01N 33/4833; G06T 7/0012; G01N 21/63; G06T 2207/10152; G01N 2333/705; G01N 2201/062; G06T 2207/30024; G06T 2207/30048; G06T 2207/10056

(57) ABSTRACT

An imaging system for imaging live biological systems comprises a detector array (12a) having an optical axis (X-X) and arranged to detect light and output detector signals, a support (10) arranged so support a biological system on the optical axis, an illuminating light source (16) located off the optical axis and arranged to direct at least partially-coherent light towards the biological system, and processing means (18) arranged to receive the detector signals and generate image data.

This invention was made with US government support under grant number HL111649 awarded by the National Institute of Health. The US government has certain rights in the invention.

### FIELD OF THE INVENTION

The present invention relates to methods, apparatus and software for optical imaging of dynamic processes in live biological systems, and to optical interrogation and imaging of such systems.

### BACKGROUND TO THE INVENTION

Researchers have been using in vitro multicellular preparations of cardiac myocytes for over 40 years to study critical tissue-level properties of the heart. In the last 15 years, these have included monolayers of cultured cardiomyocytes, derived from the native heart or differentiated from stem cells, e.g. embryonic stem cells (ESC), induced pluripotent stem cells (iPSC). When imaged at macroscopic space scales (mm and cm scales), such multicellular preparations have allowed the investigation of complex dynamic phenomena, such as wave propagation and pattern formation, and therefore are often used as a surrogate to analyze heart tissue behavior. The ability of macroscopic cardiac preparations to support spiral waves (synonymous with tachycardia) has validated their use as a model of arrhythmogenesis. High-resolution optical mapping of these preparations has given an insight into important arrhythmogenic mechanisms, including unidirectional conduction block, junctional coupling, electrical remodelling etc. Recent implementation of optogenetic means of stimulation further allows the space- and time-resolved study of dynamic processes in such multicellular systems.

Imaging of dynamic processes in live biological systems, for example as carried out on confluent myocyte monocultures or other small tissue samples, is typically dye-based (fluorescent synthetic dyes or genetically-encoded fluorescent dyes). This has many limitations, including: 1) the imaging is terminal when fluorescent dye labelling is applied due to phototoxicity, thus precluding long-term monitoring; 2) prior genetic modification is required when genetically-encoded dyes are used; 3) most current dye indicators/sensors require expensive high-sensitivity photodetectors and special focusing optics; and 4) such systems are not amenable to miniaturization, high-throughput applications and incubator long-term use.

An optical imaging method has been described by Hwang, Yea et al, 2004, in which white (non-coherent) light trans-illumination was used in conjunction with a pinhole on the axis of the imaging camera. This system required focusing optics, and only after image processing, it was possible to detect propagating waves in cardiac monolayers. As implemented, 1) this system is not easily miniaturizable; 2) it is not easily combinable with optogenetic means of actuation (due to the white light used); 3) it worked strictly in trans-illumination regime (the illuminator and the photodetector were on opposite sides of the sample); 4) it was not possible to observe propagation of waves directly (without processing); and 5) the spatial resolution was unclear.

### SUMMARY OF THE INVENTION

The present invention provides an imaging system for imaging dynamic and static processes in live biological systems. According to one aspect of the invention, there is provided an imaging system which comprises a detector array (for example, a camera) having an optical axis and arranged to detect light and to output detector signals (usable single-pixel traces), a support arranged to support a biological system on the optical axis, and an illuminating light source that may be located off the optical axis and arranged to direct at least partially coherent light towards the biological system, and processing means arranged to receive the detector signals and generate image data.

Since the effective imaging volume of the system is the volume which can be imaged by the detector array and which is illuminated by the illuminating light source, the width of the beam of the illuminating light source needs to be large enough to define an imaging volume that can contain the sample to be imaged. The illuminating light source may be arranged to illuminate the whole of the sample simultaneously. For example it may produce an illuminating beam which is wide enough to illuminate the whole of the sample holder simultaneously.

The imaging system may have a field of view (FOV), which may encompass the imaging volume, and may be arranged to detect modulation due to optical path length (OPL) changes at spatial locations across the FOV.

The system may further comprise a display arranged to receive the image data and display an image based on the image data. The image may be a video image.

The system may comprise an all-optical interrogation system where the optical imaging is combined with optical stimulation means, which may comprise a stimulation light source, arranged to direct light at the imaging volume and therefore at the biological system thereby to stimulate activity in the biological system. For example the biological system may be treated to express light-sensitive ion channels and pumps (opsins), i.e. genetically modified so as to respond to optical stimulation using light at a stimulating wavelength or range of wavelengths (complementary to the wavelength(s) used for imaging). The stimulating light source may be arranged to generate a stimulating light pattern having a number of forms. The stimulating light source may be arranged to control and vary the position or positions on the sample at which light is directed, and the time for which each position is stimulated. For example the stimulating light pattern may comprise a single pulse directed at a small area, or point, of the biological system, or sample, and lasting only few milliseconds, or it may be directed at several areas, or larger areas of the sample, and over longer periods. The stimulating light source may also generate light which is intended to suppress rather than stimulate activity in the biological system. For example it may be arranged to direct light at a large part, or substantially the whole of the biological system, or a sample holder arranged to hold the system, or an imaging volume which can be imaged by the system. That light intensity level can be set so that it simultaneously depolarizes the cells, thereby suppressing activity in the system; or it may be arranged to hyperpolarize the cells and suppress activity (with expression of proper opsins). The control system may be arranged to control the stimulating light source to generate a suppressing light pattern prior to generating a stimulating light pattern, so that the sample is inactive when the stimulating light pattern is directed at it.

The illuminating light source may be monochromatic, such as a monochromatic LED or a laser diode, or it may be another form of light source, such as a white LED or incandescent light which is band-pass filtered so as to produce a partially-coherent light source. The coherence of light sources decreases with the spectral bandwidth and with the physical size of the source. In general, the narrower the spectral bandwidth, and the smaller the illuminating light source, the more coherent the illuminating light beam will be. The spectral bandwidth can be determined by the bandwidth of the LED or other emitting element, or by a bandpass filter. This may be less than 100nm FWHM, or less than 50nm FWHM, or less than 20nm FWHM. The size of the source can be defined by the size of the LED or other emitting element itself, where essentially all of the light from the emitting element forms the illuminating beam, or by the size of aperture used to form the illuminating light beam, where some of the light from the emitting element is blocked out by an aperture to form the beam. In either case it is the size (specifically the cross sectional area) of the illuminating light beam at its narrowest point, i.e. the point where it is emitted or transmitted towards the sample. The size of the light source may be less than 10 mm² or less than 5 mm² or less than 2 mm² or less than 1 mm².

The illuminating lights source may be arranged for trans-illumination, i.e. arranged on the opposite side of the sample, or sample holder, from the detector array. This means that light received at the detector array is light from the source that has been transmitted through the sample.

The processing means may be arranged to analyze the image data to determine one or more parameters of the image data, or one or more parameters of the biological system. The parameters may comprise the presence or absence of wavefronts if the biological system comprises cardiac tissue or cells, or they may include the speed, or direction, or frequency of the wavefronts, or a measure of the shape of the wavefronts, such as radius of curvature. The processing means may be arranged to compare one or more of these parameters with one or more reference values to determine whether the biological system meets one or more criteria, and may generate an output on the basis of that determination. Real-time feedback control may be achieved as the imaging output is compared to desired reference values and the stimulation pattern is applied to bring or maintain the biological system closer to the set target, including but not limited to prevention of arrhythmia events. For high-throughput systems, this enables the system to test large numbers of biological systems and record the results for analysis.

The support means may comprise a plurality of sample holders each arranged to hold a respective sample. It may further comprise a support arranged to support each of the sample holders. The support may have a plurality of apertures through it, and may be arranged to support each of the sample holders in a respective one of the apertures. If the sample holders are transparent to the illuminating light, then the illuminating light may be directed onto the samples through the sample holders, for example from the underside.

The system may further comprise drive means arranged to move the support so that each of the sample holders can be moved into an imaging volume. The imaging volume may be the volume which can be imaged by the detector array.

The system may provide a combination of dye-free imaging of excitation waves with optogenetic stimulation in a way that directly allows scalability, high-throughput, portability, long-term noninvasive probing and observation of electrical function. It may in particular be directly applicable for high-throughput drug screening and cardiotoxicity (arrhythmia) testing, among other things. It also may be used for cell phenotyping, for monitoring and optimization of electromechanical function in patient-specific stem-cell derived cardiomyocytes, for example.

The imaging technique is understood to be based on the idea that changes in the optical path length (OPL), in the z-direction, i.e. along the optical axis of the detector array, occur upon cell excitation. Such OPL changes may be captured across millions of cells in parallel and with a sub-cellular resolution by fast imaging for example using an interferometric or phase technique. This enables, for example, image propagation of fast electromechanical waves in cardiomyocytes, that otherwise are not visible and have commonly been imaged by fluorescent techniques.

The system may be a transmitted-light optical imaging system. Some embodiments of the invention can: 1) be extremely simple and affordable, not requiring special optics and light sources, lens-free or using low-NA lenses and coherent, partially coherent, or non-coherent light sources; 2) be completely non-invasive, non-toxic, dye free imaging, allowing for repeated monitoring over days; 3) be spectrally flexible (wavelength-independent), hence easily combinable with various optogenetic actuators for truly simultaneous imaging; 4) provide fast wide-field (non-scanning) imaging, suitable for tracking intricate fast excitation waves; 5) provide ultra-high spatiotemporal resolution, revealing subcellular events at centimeter field of view; 6) enable monitoring of multiple preparations and samples simultaneously in a very time efficient manner.

The system may not require focusing (moving the preparation to a particular position on the optical axis). This omission of focusing optics may allow for straightforward miniaturization and is a significant departure from conventional imaging systems which would have to focus on different preparations individually prior to data capture. The spectral flexibility of the imaging method (practically any wavelength can be used to illuminate, unlike standard fluorescent imaging), can allow for easy combination with optogenetic stimulation, and this can allow for real time, all-optical, stimulation and monitoring of biological systems.

The biological sample may be genetically modified with optogenetic tools (light-sensitive ion channels and pumps) to become light-responsive. Space- and time-resolved optical signals of particular wavelength may be delivered dynamically by the stimulating light source to stimulate, suppress or alter activity at desired locations. One possible implementation of this is by the use of digital micromirror device to direct the stimulating light.

The system may have immediate application to the pharmaceutical industry, where the most advanced current system for high-throughput cardiotoxicity testing (FLIPR from Molecular Devices) is limited in throughput because a KCl depolarizing solution needs to be added to each well to stimulate, which is irreversible, slow, and imprecise. In embodiments of the present invention, stimulation can be done at millions of locations in parallel without contact, by light, while the biological response is instantly imaged optically.

In some embodiments, both the optical imaging and the optical actuation can be realized by simple mobile device technology, i.e. direct macroscopic imaging of excitation is possible using a cell phone camera without further optics; similarly, color videos projected from the cell phone display can be used to optically stimulate desired locations at desired times. Under certain conditions, such low irradiance can be sufficient to elicit response. The importance of this aspect is that, unlike any other technology for the study of cardiac arrhythmias, this high-throughput stimulation and imaging can be done over long periods repeatedly, remotely, in a standard incubator or custom-designed environmental chamber, without special requirements for bulky optics. This is relevant to testing of maturity and phenotype of stem cell derived cardiomyocytes over extended periods, for example.

The system may further comprise a sample holding means arranged to hold a plurality of samples and movable to move each of the samples into the field of view, or imaging volume, of the detector array in turn. The sample holding means may comprise, or be arranged to support, a plurality of sample holders. The sample holders may be transparent. The sample holding means may comprise a sample table arranged to support the sample holders. The sample holding means may be arranged to leave the undersides of the sample holders exposed. The source of illuminating light may be arranged to direct light onto the undersides of the sample holders.

The processing means may comprise a controller and may be arranged to control the sample holding means so as to move each of the sample holders into the imaging volume. The processing means may be arranged to acquire image data during each of a plurality of imaging periods, during each of which a respective one of the sample holders is arranged to be located in the imaging volume.

The invention further provides a method of imaging a biological system, the method comprising providing an imaging system having an optical axis, placing the biological system on the optical axis, illuminating the biological system with at least partially coherent light from a direction that may be inclined to the optical axis, and imaging the biological system.

The biological system may be a monolayer or thin layer of cells, for example less than lmm thick, or less than 100 μm thick. The layer may be sufficiently thin for a detectable proportion of the light to pass through it. for example cardiac cells, in particular optogenetically modified cardiomyocytes or myocytes mixed with other cell types, where cell-specific optical stimulation may be possible by genetically modifying different cell types to respond to different wavelengths.

The method may comprise optically stimulating the biological system, for example using a pulse of stimulating light, at one or more points. The stimulation may be done prior to or during the imaging.

The method may further comprise optically suppressing activity of the biological system, for example prior to, during or after the stimulation.

The method may further comprise generating image data from the imaging system or a part thereof, and analyzing the image data, for example to determine whether the image data, or the biological system, meets one or more criteria.

According to a further aspect of the invention there is provided an imaging and control system for imaging and controlling live biological systems, the system comprising a detector array arranged to detect light from a field of view and output detector signals, a support arranged so support a biological system in the field of view, an illuminating light source arranged to direct light towards the biological system, processing means arranged to receive the detector signals and generate image data; and optical stimulation means arranged to direct light at the biological system thereby to stimulate activity in the biological system.

The processing means may be arranged to control the optical stimulation means, and to vary its optical output in response to the analysis of the image data. The processing means may be arranged to vary said optical output while the imaging system is imaging the biological system. The system may thereby provide real-time control of activity of the biological system.

According to a further aspect of the invention there is provided a method of imaging and controlling an optogenetic biological system, the method comprising providing an imaging system, placing the biological system in the field of view of the imaging system, optically stimulating or suppressing activity in the biological system using a first light source, illuminating the biological system using a second light source, and imaging the biological system. The optical stimulation or suppression of activity in the biological system, the illumination of the biological system and the imaging of the biological system may be performed simultaneously. For example the imaging of the biological system may be performed over an imaging period, and the stimulation or suppression may be varied during the imaging period to provide real-time control of the activity.

Some areas of applicability of the present invention include:
High-throughput drug screening—since it can offer massive parallelization at low cost and with new functionality (the ability to stimulate and image optically at millions of locations);
Phenotype screening of stem-cell derived cardiomyocytes (CMs), arrhythmogenicity index;
Active control of phenotype in (stem-cell derived) cardiomyocytes or other cell types;
Patient-specific drug testing using stem-cell derived CMs;
Automation and remote control of laboratory experiments.

The system may further comprise any one or more features, in any workable combination, of the embodiments of the invention which will now be described by way of example only with reference to the accompanying drawings.

### BRIEF DESCRIPTION OF THE DRAWINGS

FIG. 1 is a schematic view of an imaging and control system according to a first embodiment of the invention;

FIG. 2 is a schematic side view of an imaging and control system according to a second embodiment of the invention; and

FIG. 3 is a plan view of the system of FIG. 2;

FIG. 4 is a schematic side view of an imaging and control system according to a further embodiment of the invention;

FIG. 5 is a top view of part of the system of FIG. 4;

FIG. 6 is a schematic side view of a system comprising several of the systems of FIG. 4 housed in a single incubator;

FIGS. 7 and 8 show two successive image frames showing the presence of a re-entrant spiral wave of activity in a monolayer culture captured using a system similar to that of FIG. 1;

FIGS. 9a to 9c show intensity vs time plots for the central 5x5 pixels for the data set of FIGS. 7 and 8: FIG. 9a shows intensity from unprocessed images and FIGS. 9b and 9c show the improved s/n after processing, with FIG. 9c showing the intensity vs time plot from 1 pixel, or a 6.5 p.m area on the monolayer;

FIG. 10 is a schematic diagram of the optical arrangement of some embodiments of the invention;

FIG. 11 is a schematic diagram of an imaging system forming part of a further embodiment of the invention;

FIG. 12 is a schematic diagram of an imaging system forming part of a still further embodiment of the invention;

FIG. 13 is a schematic view of an optical stimulation and imaging system according to a further embodiment of the invention;

FIG. 14 is an image generated by the system of FIG. 13;

FIG. 15 is a further image generated by the system of FIG. 13;

FIG. 16 is a plot of intensity at one pixel of the images of FIGS. 14 and 15 as a function of time during pulsed optical stimulation of the sample;

FIGS. 17 and 18 are activation maps obtained from the system of FIG. 13;

FIG. 19 is a plot of intensity as a function of time for two pixels in the images of FIGS. 14 and 15 showing transient activity;

FIG. 20 is an image generated using the system of FIG. 13;

FIG. 21 is an enlargement of part of the image of FIG. 20;

FIG. 22 is a plot of intensity as a function of time for a responsive pixel and a non-responsive pixel in the image of FIG. 21;

FIG. 23 shows various steps in an image processing method used to analyse data from the system of FIG. 13;

FIG. 24 shows plots of intensity as a function of time for one pixel of an image generated using the system of FIG. 13, for different light sources and different distances from the focal plane; and

FIG. 25 is a plot showing variations in signal to noise ratio as a function of defocus for the plots of FIG. 24.

### DESCRIPTION OF THE PREFERRED EMBODIMENTS

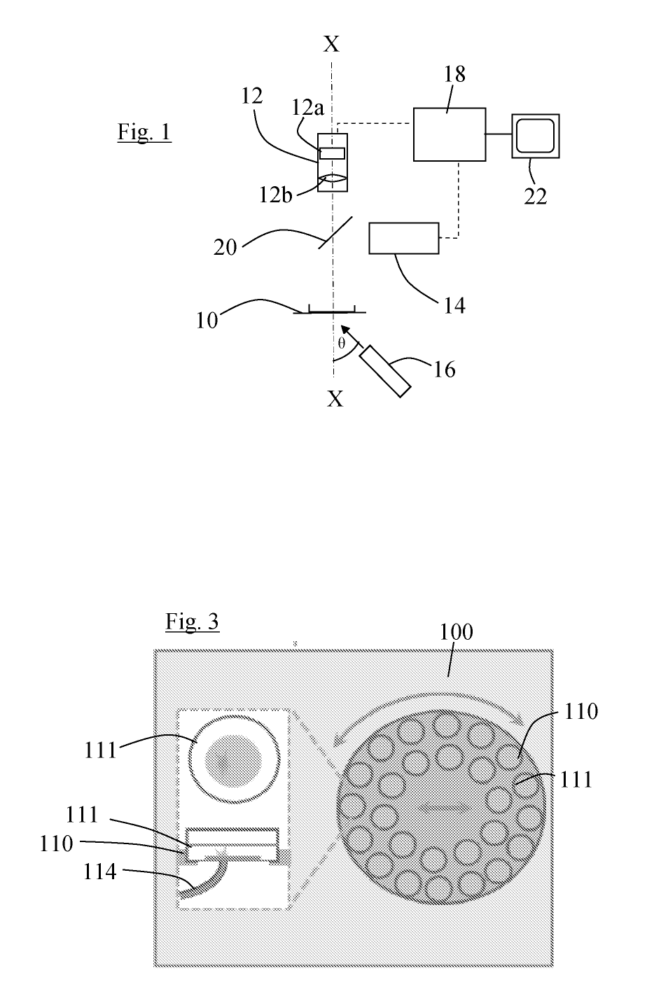

Referring to FIG. 1, an imaging system according to an embodiment of the invention comprises a sample holder 10, and a camera 12 arranged above the sample holder 10 with its axis X-X, extending, in this case, vertically downwards, through the centre of the sample holder 10. The camera comprises a detector array 12a and a lens system 12b. The axis X-X of the camera extends through the centre point of the detector array and perpendicular to the plane of the detector array. The lens system can be very simple and may be non-adjustable so that the imaging system cannot be focussed. The focal plane of the imaging system, for example if it comprises a simple objective lens, may be above or below the sample holder 10. A stimulating light source 14, for example in the form of a light projector, is arranged to project images or light patterns onto a dichroic mirror 20 arranged obliquely on the optical axis of the camera 12, between the camera 12 and the sample holder 10, so that the images are reflected down onto the sample holder 10. The mirror 20 is arranged to transmit light of an illuminating wavelength, different from the wavelength of the stimulating light, from the sample holder 10 to the camera 12 so that the camera can image the sample while the projector is projecting light onto the sample. The system further comprises an illuminating light source, in this case in the form of a source of at least a semi-coherent light 16, such as a monochromatic LED light source, which is located, in this case, below the sample holder 10, and is arranged to direct light onto the sample holder from an illumination direction which is off the axis X-X of the camera 12 so that light from the light source 16 illuminates the whole, or at least a substantial part of, the sample holder, but does not saturate the camera 12. Other partially or fully coherent light sources, such as lasers, can be used as the illuminating light source.

A control system 18 is connected to the projector 14 and is arranged to control it so as to control the projection of light onto samples in the sample holder 10. The dichromic mirror 20, which is located on the optical axis X-X of the camera between the camera and the sample holder, is arranged to direct light from the projector onto the sample. The projector 14 is arranged to transmit light from any combination of one or more points or light sources, which can be individually activated or de-activated, so that light can be projected simultaneously onto any combination of points on the sample holder, and consequently onto any combination of points on a sample that is held in the sample holder. The control system 18 can therefore control the projector so as to illuminate any area or combination of areas of the sample on the sample holder.

The control system 18 is also connected to the camera 12 and is arranged to receive and process detector signals from the detector array 12a of the camera and to generate image data from the signals. It is further arranged to process the image data using image processing algorithms to improve the image data. The control system 18 is also connected to a display 22 and arranged to use the image data to control the display 22 to display images of the sample. The images may be real time video images. The control system may be arranged to analyse the image data, for example as described below with reference to the second embodiment.

It has been found that if multicellular preparations of optogenetically modified cardiomyocytes, produced for example using channelrhodopsins, are placed on the sample holder 10, and stimulated with light, for example at a point or line, or series of points or lines, the activation wavefronts that travel through the sample can be seen in real time in the images with only basic background subtraction image processing algorithms. The images are therefore not simple transmission images, but involve a degree of refraction and/or interference of the illuminating light.

It has been found that good contrast and signal-to-noise ratio are achieved when the illuminating light source is arranged to direct the illuminating light onto the sample from an illumination direction at an angle θ of at least 5°, and typically at least 10° off the camera axis X-X, and preferably no more than 80° off axis, where an angle of zero corresponds to the light being along the axis towards the camera. This angle depends on the detector optics or lack thereof, i.e. wider angle may be needed for imaging optics with higher numerical aperture. Illumination at small angles, including zero, is possible when combined with other imaging methods, for example as described below with reference to FIGS. 11 and 12. However, with the particular implementation of trans-illumination off-axis of the embodiments described above, there is no need for a pinhole or a reference beam since all light needed to produce the image comes from the illuminating light source, and any interference is produced by that light interacting with the biological system components. Other system solutions are also applicable to achieve such dynamic macroscopic imaging, as will be described in more detail below.

It has been found that the imaging system is capable of very high resolution and may be diffraction-limited. In macroscopic imaging, with FOV in the centimetre range, the spatial resolution of the detector may be the limiting factor, i.e. single pixel (6.7 μm) in 1× imaging was found to give informative signals without the need for binning.

It has also been found that, with this imaging system, good images can be obtained even if the optical system 12b of the imaging system is not focussed on the sample. For example the optical system 12b may be arranged to focus an image of the sample either in front of or behind the detector array 12a. This also means that the image which is focused on the detector array is of a region above or below the sample holder 10. This means that a very simple focusing optics, or indeed no focusing optics, can be used and the activity in the sample can still be imaged and observed clearly in real time.

A possible explanation for the imaging of the system is that the detector array is arranged to detect interfering light that has travelled along different paths having been refracted at different parts of the sample, with the difference in optical path length dependent on the activity in the sample. A further possible explanation is that the contracting cells are optically more dense, and therefore scatter light more effectively, and this gives rise to a brighter signal above contracted cells (since all the zero-order light is pointed away from the camera), so the detector will detect the wavefront which is then visible in the image. A further possible explanation is that the cells act as optical elements, and when they contract they refract light toward the sensor. In this case the relaxed cells would be acting like a flat piece of glass and simply transmit light, and the contracted cells would act as a lens and refract it. A still further possible explanation is that the internal structure of the myocytes acts as a diffraction grating, producing lines of constructive and destructive interference forming a diffraction pattern, so that when the cells are relaxed the lines in the diffraction pattern are far apart, and when they contract they are close together. Since the illuminating light source is off axis, then more light is directed to the camera when the lines are far apart (during contraction).

Using this system, like in other imaging modalities, in particular holographic modalities, it is possible, with knowledge of the optical system and optical paths and inclusion of a reference beam, to reconstruct information about simultaneous activity from multiple planes within the biological system (to achieve 4D imaging in three spatial dimensions and over time) using post-processing on single recording (without moving through different planes).

Therefore the control system 18 may be arranged to control the stimulation and imaging of the sample. For example the control system may be arranged to generate a stimulating pulse of light directed at one point on the sample holder (and hence the sample) for a short stimulation period, and the camera 12 may then be arranged to capture a video image over an imaging period immediately following the stimulation period or concurrent with the stimulation or after the stimulation, for a period that may last for example from 5 to 20 seconds.

The control system 18 may also be arranged to control the projector 14 to generate a light pattern intended to suppress rather than stimulate activity in the biological system. For example it may be arranged to direct light at a large part, or substantially the whole of the sample holder 10. That light can be stronger, i.e. of a higher intensity, that the light needed for stimulation (when the biological systems is modified by depolarizing channelrhodopsins) or it may be of a wavelength suitable for suppressing activity in the system (when the biological system is modified by hyperpolarizing opsins, including but not limited to eNpHR3.0 (chloride pumps) or iC1C2 (chloride channel) or ArchT (H+ pump) or Jaws, which may be different from that of the stimulating light pattern. Bi-directional control (stimulation and suppression) may be applied with the combined expression of depolarizing and hyperpolarizing opsins and two different wavelengths.

The control system 18 may be arranged to provide real time control of the activity in the biological system. For example, during the imaging period the control system may be arranged to identify, from the image data, one or more features of the imaged biological system, and to monitor how the one or more features varies over time. For example the control system may be arranged to determine the location of activity within the biological system, or the speed or direction of movement, or the shape, of patterns of activity in the biological system. The patterns of activity may be wave fronts in cardiac tissue. The control system may then be arranged to monitor those parameters and to determine whether they are within predetermined values or limits, which may be stored in memory in the control system. If the control system determines that the parameters are outside those values or limits, it may be arranged to vary the stimulating light source, so as to vary the stimulating and/or suppressing light patterns it generates, thereby to maintain the activity in the desired parameter ranges, for example in the desired location.

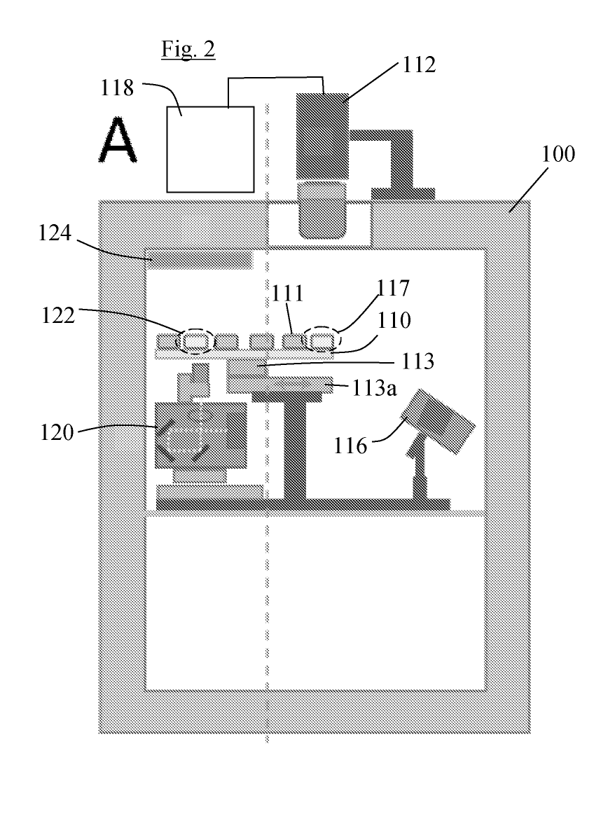

Referring to FIGS. 2 and 3, a system according to a second embodiment of the invention comprise an incubator 100 having a sample table 110 arranged to carry a number of samples in a number of sample holders 111, for example petri dishes. The sample table 110 has a number of holes through it each with a lip around the bottom so that a sample holder can be place in each hole, supported on the lip, with its underside exposed by the hole. The sample table 110 is rotatably mounted on a rotary drive mechanism 113 which in turn can be moved horizontally by a lateral drive mechanism 113a. A camera 112 is arranged in a window 115 in the top of the incubator 100, above the sample table 110 with its axis extending vertically downwards. The camera comprises a detector array 112a and a lens system 112b. The lens system is in this case simply adjustable to provide a degree of focusing, but could be completely fixed. The system further comprises a source of at least semi-coherent light, 116 such as an LED light source, which is located, in this case, below the sample table 110, and arranged to direct light onto, and thereby illuminate, an imaging volume 117 which is on the axis of the camera 112 and in the plane level with the sample holders 111. As the illuminating light source 116 is below the sample table 110 it is arranged to direct light upwards though the hole in the sample table 110 into whichever sample is located in the imaging volume. The sample holders 111 are transparent so that the illuminating light can pass through them into the samples. The rotational and horizontal adjustment of the sample table 110 allows each of the sample holders 111 to be moved into the imaging volume 117 so that it can be illuminated by the light source 116 and imaged by the camera 112. The illuminating light source 116 can again be arranged off the camera axis so as to direct light onto the samples from an illumination direction which is off the axis X-X of the camera 112. A stimulating light source in this system comprises a fibre optic cable 114 arranged to direct semi-coherent light upwards towards the underside of the sample table at a point on the axis of the camera 112 and therefore within the imaging volume 117.

A processor 118 is connected to the camera 112 and arranged to receive and process the raw detector signals from the camera. The processing may be as described above with reference to FIG. 1, or may take other forms as described in more detail below. In particular it may be arranged to analyse the image data and determine one or more parameters of the image data, or of the sample. It may then be arranged to determine whether it meets one or more criteria, for example by comparing the parameters with reference values stored in memory. It may then be arranged to generate an output indicative of whether each of samples, or each of the images of a respective one of the samples, meets those criteria. Alternatively it may simply store the parameters for later analysis.

The parameters that processor 118 is arranged to determine may comprise the presence or absence of wavefronts if the biological system comprises cardiac tissue or cells, or they may include the speed, or direction, or frequency of the wavefronts, or a measure of the shape of the wavefronts, such as radius of curvature. The processor 118 may be arranged to compare one or more of these parameters with one or more reference values to determine whether the biological system meets one or more criteria, and may generate an output on the basis of that determination. Real-time feedback control may be achieved as the imaging output is compared to desired reference values and the stimulation pattern is applied to bring or maintain the biological system closer to the set target, for example to prevent of arrhythmia events. For high-throughput systems, this enables the system to test large numbers of biological systems and record the results for analysis.

The system also comprises a microscopic imaging system 120 arranged to form microscopic images of the samples in a separate imaging volume 122, and a macroscopic imaging system 124 also arranged to image samples in the imaging volume 122.

The controller 118 is connected to the rotary and lateral drive mechanisms 113, 133a for the sample table 110 and is arranged to move the table so that each of the position above each of the sample apertures in turn is located in the imaging volume 117, and held there for a predetermined imaging period, before the table is moved again and the position above the next aperture is located in the imaging volume. This moves each of the samples into the imaging volume in turn. During each imaging period, the controller 118 is arranged to stimulate the sample in the imaging volume by activating the stimulating illumination from the fibre optic cable 114, and then to acquire video image data from the camera 112 over the course of the imaging period. It is then arranged to store the image data for analysis and display.

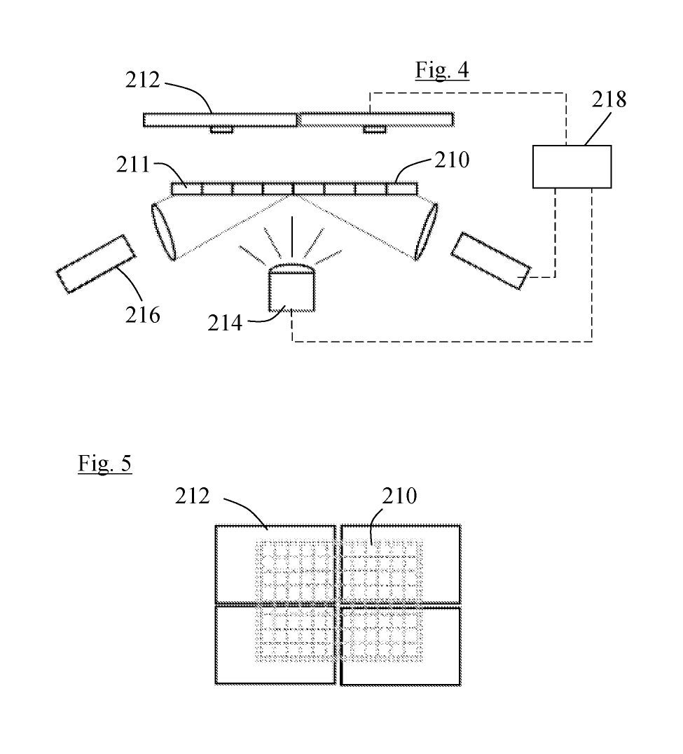

Referring to FIGS. 4 and 5, in a third embodiment of the invention, a group of four camera devices 212, which in this case are mobile phones with cameras, are arranged over a standard 96 well plate 210, so that each camera device 212 images a respective group of 24 of the wells 211 each of which holds a respective sample. Two illuminating light sources 216, which again can be LEDs or laser diodes, are arranged to direct illuminating light simultaneously at the undersides of all of the wells 211. A further LED light source 214, of a different wavelength from the illuminating light source, is provided for optogenetic stimulation of samples located in the wells 211. These may be preparations with opsins. In this embodiment the stimulating LED 214 provides uniform illumination to stimulate all samples in the wells 211. The camera devices 212 are each connected to a central control unit 218, which may be in the form of a suitably programmed PC, and the illuminating light sources 216 and stimulating light source 214 are also connected to the control unit 218. The control unit 218 is arranged to control the stimulating light source 214 so as to control the timing and intensity of the illuminating light that it generates. This may be in the form of pulses, in which case the start times, duration, and intensity of each of the pulses may each be controlled. The control unit 218 is also arranged to receive image data from the camera devices 212, either streamed during acquisition or as image data files after image acquisition is complete.

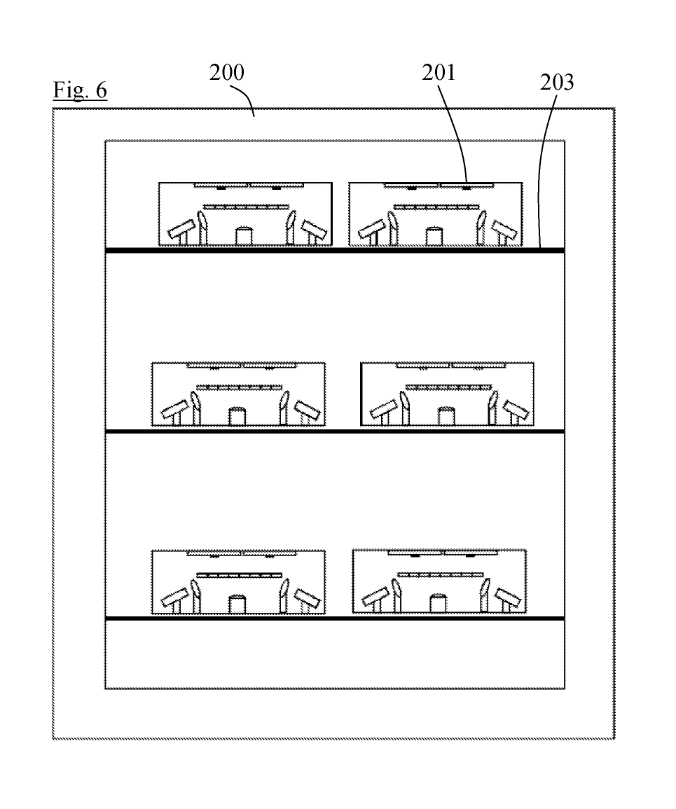

The system of FIGS. 4 and 5 is very compact and simple, and, referring to FIG. 6, a number of such systems 201 are, in one embodiment, mounted within a standard incubator 200 on shelves 203. In this case each of the systems 201 can be connected to the same control unit. This provides a simple high throughput system that can be used for controlling and imaging a variety of different types of biological system under controlled conditions.

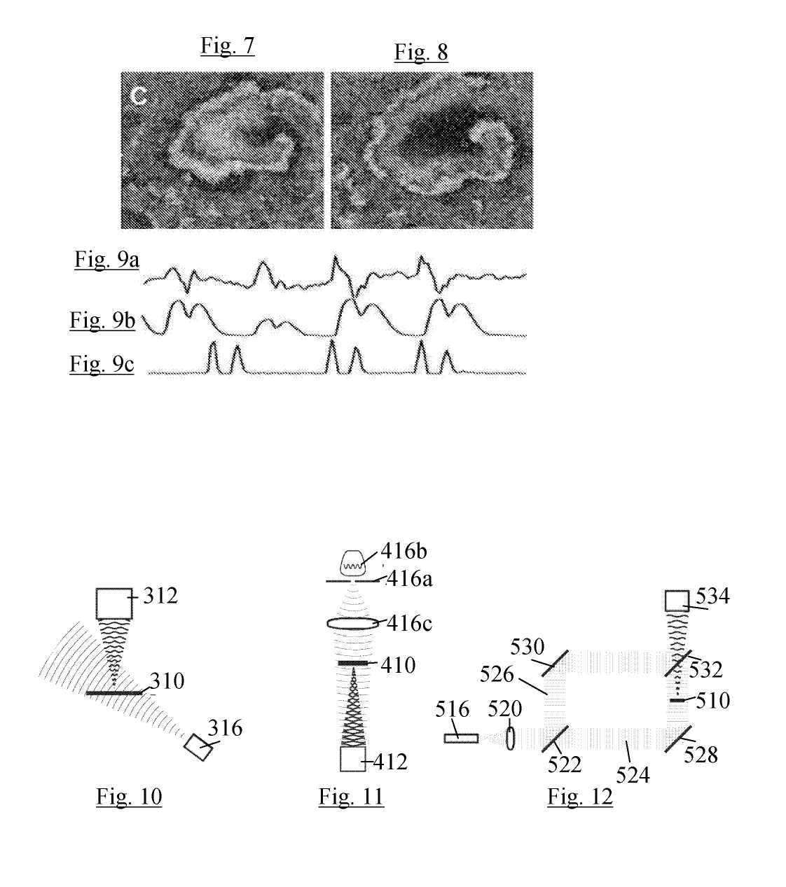

Referring to FIGS. 7 and 8, a system based on FIG. 1, in which the illuminating light source was a green light emitting diode (Cairn Research OptoLED light source), the camera was an Andor Neo 5.5 sCMOS, was used to produce images of a monolayer culture of neonatal rat cardiomyocytes. FIG. 9a shows intensity from unprocessed images and FIGS. 9b and 9c show the improved s/n ratio after processing, with FIG. 9c showing the enhanced intensity vs time plot from 1 pixel, or a 6.5 μm area on the monolayer.

Referring to FIG. 10, each of the embodiments described above includes, in general terms, an imaging device or system 312, and an off-axis illuminating light source 316, for imaging a sample in a sample holder 310. However, the combined optical stimulation and imaging provided by those embodiments can also be provided using different types of imaging system. For example, referring to FIG. 11, in a further embodiment the illumination of the sample holder 410 is provided by a pinhole aperture 416a, illuminated by a light source 416b, which can be a simple white incandescent lamp, optionally with a lens 416c provided between the pinhole 416a and the sample. The imaging is performed by a camera device 412, with the sample and the pinhole being located on the axis of the camera device 412. The stimulating light source is not shown in FIG. 11, but can be a simple light source similar to that of FIG. 4 with a control unit arranged to control the timing and intensity of the light, but not the position, or it can be a more complex projector light source similar to that of FIG. 1 with a control unit arranged to control the position and timing and optionally also the intensity of the stimulating (or suppressing) light.

Referring to FIG. 12, a further embodiment of the invention comprises an imaging system having a Mach-Zehnder interferometric setup. This comprises a light source 516 with a lens 520 forming an illuminating beam, a beam splitter 522 arranged to split the beam into a main beam 524 and a reference beam 526, mirrors 528, 530 arranged to reflect the main beam 524 and the reference beam 526 onto a further beam splitter 532, and a photodetector array 534 arranged to detect the re-combined beams. A sample holder 510 is located in the path of the main beam, between the mirror 528 and the second beam splitter 532. As with the embodiment of FIG. 11, a stimulating light source (not shown) is arranged to stimulate activity in a sample in the sample holder and can take any of the forms described above.

Each of the systems of FIGS. 11 and 12 can provide all-optical stimulation/suppression and imaging of the sample providing real time control and interrogation in a similar way to the previous embodiments.

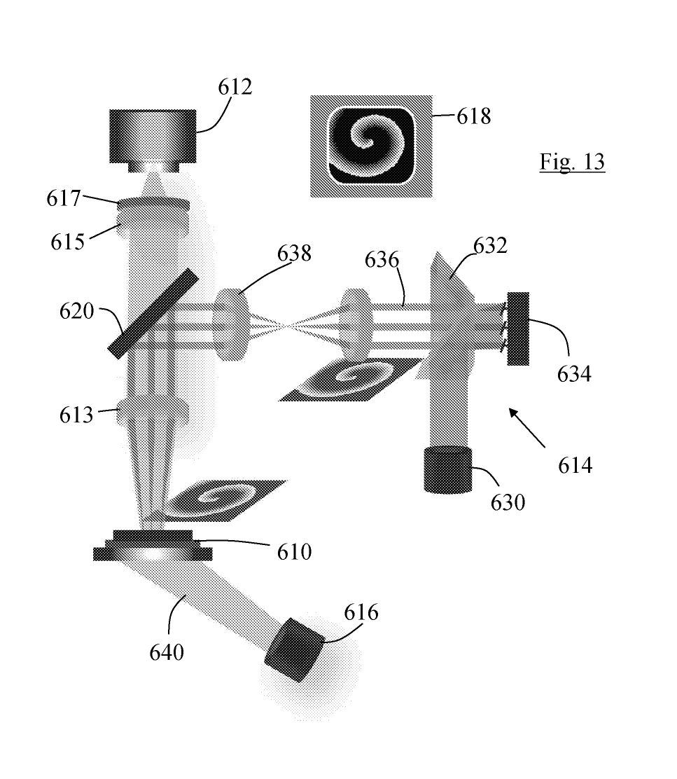

Referring to FIG. 13 an optical control and imaging system according to a further embodiment of the invention comprises a sample holder 610, a camera 612 mounted above the sample holder 610 and arranged to image samples on the holder 610, a stimulating light source 614, an illuminating light source 616 and a computer 618. The camera 612 is a scientific complementary metal-oxide semiconductor (sCMOS) camera. An objective lens 613, an imaging lens 615, and a long-pass emission filter 617 are arranged between the sample holder 610 and the camera 612, on a common axis with the centre of the camera 612 and the centre of the sample holder 610 which forms the optical axis of the imaging system, and a dichroic mirror 620 is located on the optical axis and inclined at 45° to it, between the objective and imaging lenses 613, 615. The dichroic mirror is arranged to reflect light with wavelength less than a threshold wavelength, in this case 510 nm, and to transmit light of higher wavelengths. The stimulating light source 614 comprises a source of semi-coherent light in the form of a 10W LED 630 arranged to output light of wavelength 460 nm, a total-internal-reflection prism 632, and a digital micromirror device (DMD) 634 which is controlled by the computer 618. The LED 630 is arranged to direct light onto the prism 632 which is arranged to reflect it onto the DMD device 634. The DMD device is arranged to reflect some of the light, in a pattern that is controlled by the computer 618, as a patterned stimulating light beam 636, back through the prism 632, through a further pair of lenses 638 onto the dichroic mirror 620, which reflects the stimulating light beam down onto the biological sample which is on the sample holder 610. The illuminating light source 616 is located below the sample holder 610 and off the optical axis of the imaging system, and comprises a white LED, bandpass filtered at 580 ±20 nm. It is arranged to generate an illuminating light beam 640 directed at the underside of the sample holder. The sample holder 610 is transparent to light at the wavelength of the illuminating light source 616, which therefore provides oblique trans-illumination of any sample placed on the sample holder.

The system of FIG. 13 was used to stimulate and image a genetically modified cardiac monolayer, which was placed on the sample holder 610. The computer 618 was arranged to control the stimulating illumination to provide over a period P1 a uniform stimulating pulse P1 of about is in length, followed by a sequence, over period P2, of periodic line stimuli each of which illuminated a line across the centre of the sample, at pulse frequency of about 2 pulses per second.

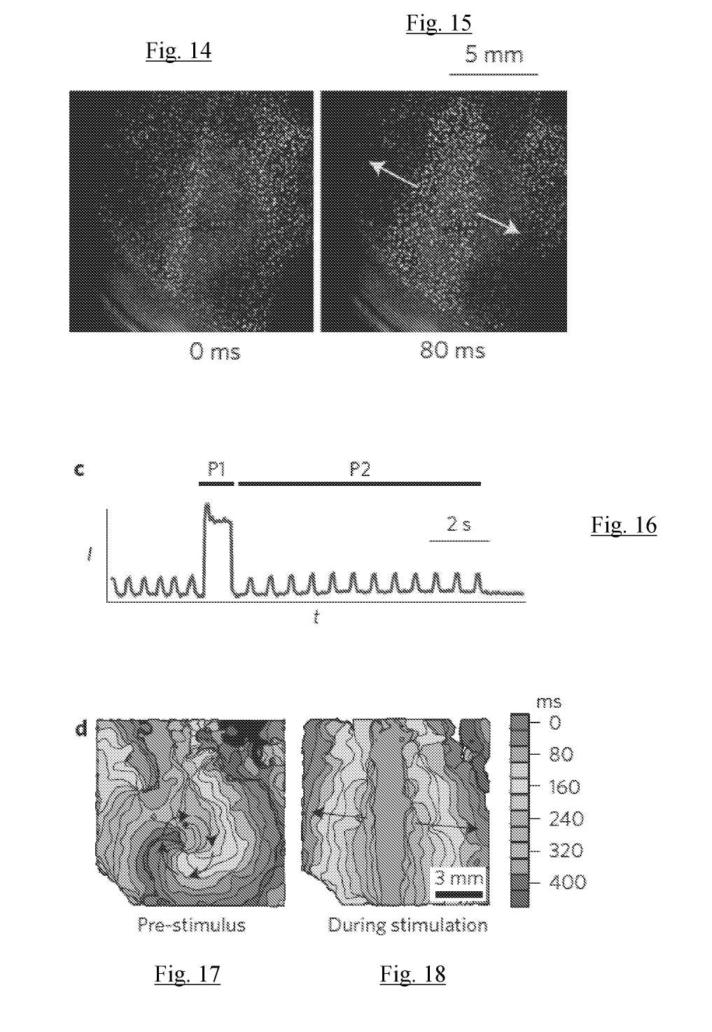

FIGS. 14 and 15 are examples of minimally filtered images of the sample responding to the pulsed optical line stimulation, at the time of one of the pulses, and 80ms after the pulse.

FIG. 16 is a trace of intensity (I) versus time (t) from a single pixel in the images of FIGS. 14 and 15, indicating the time of the high intensity pulse P1 and the periodic linear pulses P2.

FIGS. 17 and 18 are activation maps showing ongoing spontaneous activity (a spiral) pre-stimulus, and, following termination of the spiral activity by a high intensity light pulse applied over the entire field, generation of a planar wave by application of a linearly patterned light pulse.

In some cases, excitation waves are visible without image processing. Visibility of excitation waves was enhanced during live recording by applying a running background subtraction followed by an absolute value operation on each pixel:

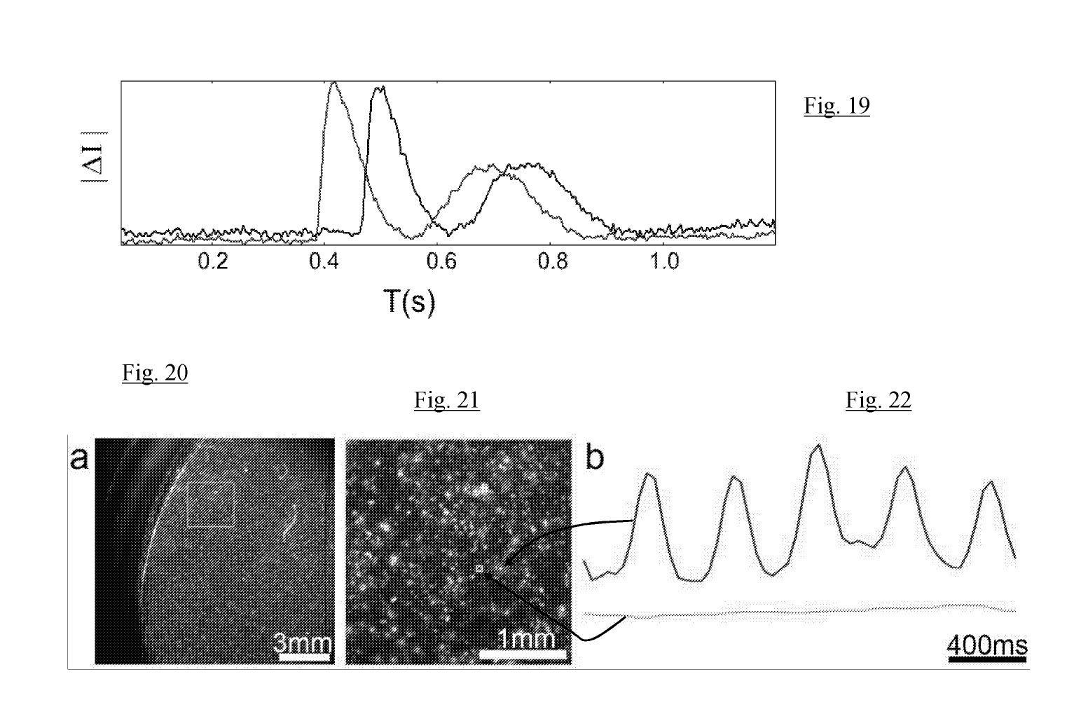

Pₜ(i,j)=|pₜ(i,j)−pₜ₋ₙ(i,j)| (1)

Where pₜ(i, j) is the value of pixel p at location i,j at time t, and pₜ₋ₙ(t,j) is the value of the same pixel at an earlier time point (typically 120 ms, or 6 frames apart, where n is the number of frames). The low computational overhead of this filter operation allowed assessment of activity in real time. An example of the output of this operation is the two frames in FIGS. 14 and 15. Plotting the filtered intensity vs. time curves for the data yields a doubled humped curve, where the first spike corresponds to activation and the second hump corresponds to relaxation as shown in FIG. 19, which shows curves for two pixels located at different points in the path of a travelling wave front. Data filtered in this fashion can be directly used for analysing simple wave dynamics (e.g. calculation of conduction velocity for planar waves). However, interpretation of complex spiral and wavelet patterns were hindered by the presence of the second hump, as it was often difficult to distinguish excitation from relaxation waves. A different method was therefore used to measure the shape of excitation waves for generating the activation maps of FIGS. 17 and 18.

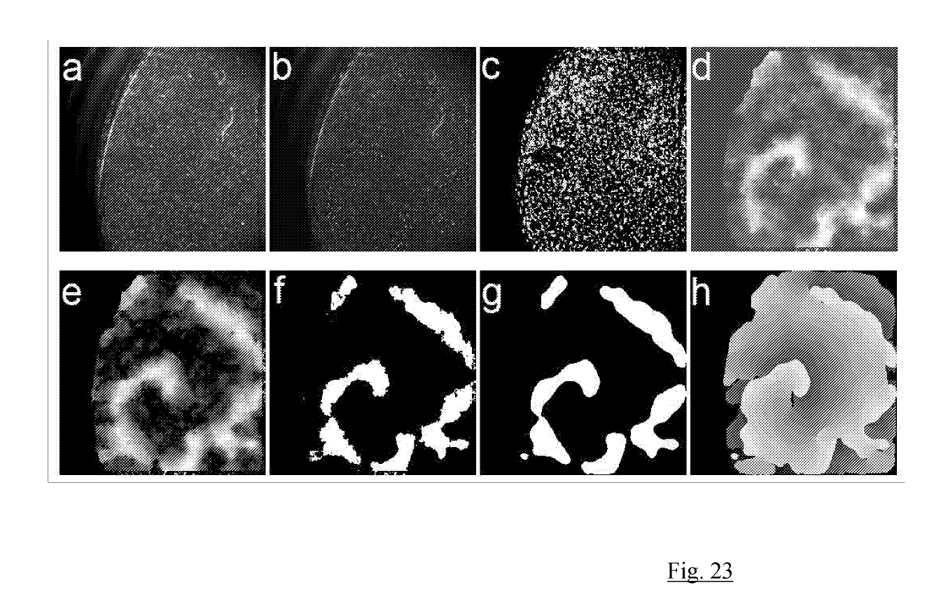

Measurement of activation wave patterns as shown in FIGS. 17 and 18 was achieved as follows. After acquisition, a running background subtraction (where each pixel at frame t is replaced by its value minus the average value of that pixel over 2n+1 frames between t−n and t+n), where n can be selected as appropriate, gives a clear image of wave dynamics. The dichroic mirror and band pass filters pass a small amount of excitation light so that the imposed illumination patterns may be directly visible in this record. Referring to FIGS. 20 and 21 raw data consists of high contrast regions distributed throughout the sample. FIG. 20 is a full field of view single frame. The edge of the 35mm petri dish is clearly visible. FIG. 21 is a full resolution zoom showing bright and dark regions. FIG. 22 shows intensity vs. time plots from two pixels in FIG. 21, as indicated by the arrows, which show markedly different transients. Bright regions in the image, as shown in the upper plot, are more likely to show activity related to contraction of the monolayer than dark regions, as shown in the lower plot.

Quantification of wavefront location was performed in several steps. Off-axis illumination results in localised regions of high contrast as can be seen in FIGS. 20 and 21. These regions typically display an increase in intensity that corresponds to contraction, as shown in the upper plot in FIG. 22, while darker regions do not display transients that can be distinguished from noise, as shown in the lower plot of FIG. 22. The data sets generated from this imaging technique yield a sparse set of points that can be used to track the continuous contraction waves in the underlying tissue.

One method of data analysis which can be used to obtain the wavefront patterns will now be described. Images are first spatially binned by a factor of 8 prior to processing in order to reduce computation time, the data from one binning being shown in FIG. 23b. The data is then scaled between 0 and 1, and all pixels with average value less than 0.1 are classified as nonresponsive and all pixels with average value equal or greater than 0.1 are classified as responsive. These are shown as black and white pixels in FIG. 23c. Nonresponsive pixels are replaced by the average of those responsive pixels within a radius of 8 (binned) pixels for each frame. After this operation, responsive pixels are also spatially averaged (radius 8) the result of which is shown in FIG. 23d. Intensity as a function of time traces for each pixel are then cross correlated against a single transient from a representative active pixel. The value of the cross correlation is then used to generate a data set that is used to locate wavefronts as shown in FIG. 23e. Isochronal maps, which show the location of the wave front over several frames as in FIG. 23h are generated by performing a threshold operation (pixel intensity>50% maximum) on the cross correlation data sets, as shown in FIG. 23f, and replacing the active pixels with a time stamp. Sequential frames are collapsed into one frame so that time of activation values overwrite existing values if the difference between them is greater than 50 ms (the minimum refractory period), as shown in FIG. 23h. Isochronal lines are found using a marching squares algorithm, and colour coded using the ‘rainbow smooth.lut’ lookup table.

It will be appreciated that the image of FIG. 23h shows the progress of the wave fronts over time, and that parameters of the shape of the wave fronts, such as radius of curvature, can be extracted from this type of image, and that the speed and direction of the wave fronts can also be determined from the relative positions of the boundaries of the different areas in the image.

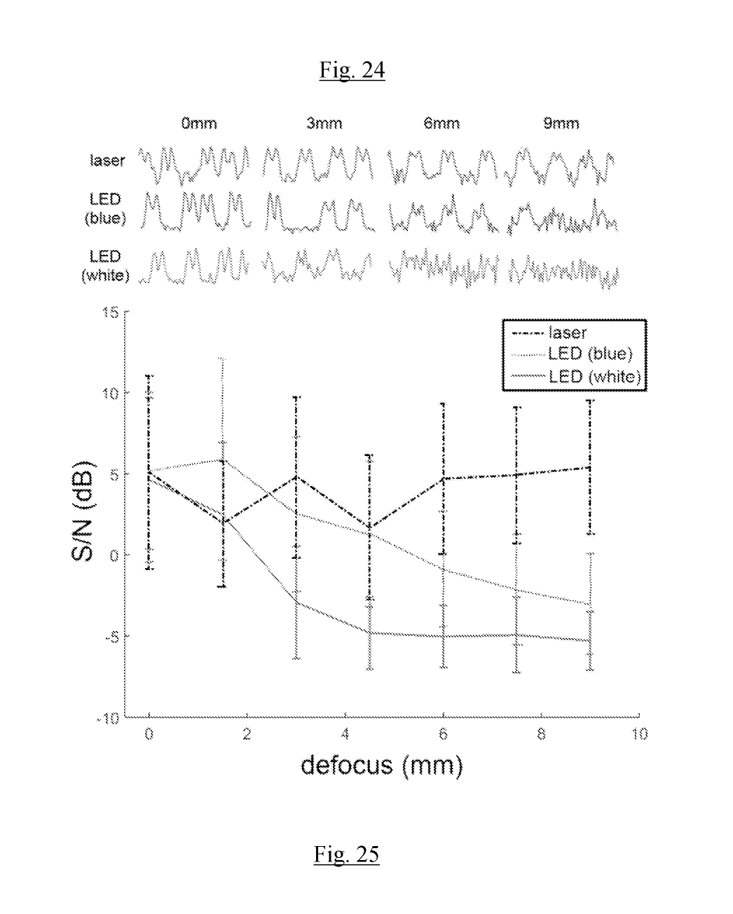

Referring to FIGS. 24 and 25, the performance of the imaging system were experimentally characterised using three light sources which differed in their spatial and temporal coherence: a white LED, a blue LED (Cairn Research), and a red laser (Thorlabs CPS186), imaging spontaneously active samples. The three light sources were held within the same optical mount, and a series of images were captured at different focal planes (every 1.25 mm) using a mechanical focus motor (Prior Scientific H22) to move the objective to ensure that the image series can be directly compared. The image contrast was assessed for the three light sources by measuring the signal to noise ratio (Matlab ‘snr’ function) of time-varying intensity (raw image data was first filtered using Eq. 1 above), extracted from 64 equidistant regions of interest within 200 frame image sequences. Light sources were adjusted so that waves were visible at the focal plane. When in perfect focus, all three sources can image wave activity and show strong spikes in the intensity vs time plots (SNR>5 decibels). When defocused, the white LED loses signal rapidly, while the more coherent blue LED maintains signal over several millimetres. The difference in signal quality is significant for the LED sources (P<0.01, t-test and Wilcoxon ranksum) for all defocus values over 0. The laser, with the highest coherence, maintains a high SNR over the entire defocus range. This dependence of image quality on the spectral bandwidth (coherence) of the light source is indicative of likely interference contribution to the image formation.

Many of the described applications are for fast excitation waves in excitable tissue, e.g. cardiac tissue, but the invention is applicable to other dynamic processes, which can be optically manipulated (including but not limited to molecule signalling, pH changes, optically-controlled gene expression changes etc.) and can result in OPL changes to be imaged with the described dye-free system.

Indeed, while specific embodiments of the invention have been described, the skilled man will readily appreciate that modifications to any one or more features of these embodiments can be made as appropriate to make the system suitable for a variety of applications.

1.-34. (canceled)

35. An imaging system for imaging live biological systems, the system comprising a detector array having an optical axis and arranged to detect light received at the detector array and output detector signals, a support arranged to support a biological system on the optical axis, an illuminating light source located off the optical axis and arranged to direct at least partially-coherent light towards the biological system from an illumination direction which is off the optical axis, and a processor arranged to receive the detector signals and generate image data.

36. A system according to claim 35 wherein the illumination direction is at least 5° and no more than 80° off the axis.

37. A system according to claim 35 wherein the system is a transmitted-light optical imaging system in which the light received at the detector array is light from the source which has been transmitted through the biological system.

38. A system according to claim 35 further comprising a stimulating light source arranged to direct light at the biological system thereby to stimulate activity in the biological system.

39. A system according to claim 38 wherein the stimulating light source is arranged to generate stimulating light patterns having a plurality of different forms, thereby to stimulate different areas of the biological system.

40. A system according to claim 38 wherein the stimulating light source is further arranged to generate light arranged to suppress activity in the biological system.

41. A system according to claim 40 wherein the stimulating light source is arranged to generate light of two different wavelengths, one of which is for stimulating activity and the other of which is for suppressing activity.

42. A system according to claim 40 wherein the stimulating light source is arranged to generate light of two different intensities, one of which is for stimulating activity and the other of which is for suppressing activity.

43. A system according to claim 35 wherein the processor is arranged to analyze the image data to determine one or more parameters of the image data, or one or more parameters of the biological system.

44. A system according to claim 43 wherein the processor has access to one or more reference values and defines one or more criteria, and is arranged to compare one or more of the parameters with the one or more reference values to determine whether the biological system meets the one or more criteria, and to generate an output on the basis of that determination.

45. A system according to claim 35 wherein the support comprises a plurality of sample holders each arranged to hold a respective biological system;
the sample holders are transparent and the light source is arranged to direct the illuminating light through the sample holders; and
the system defines an imaging volume and further comprises a drive mechanism arranged to move the support so that each of the sample holders can be moved into the imaging volume.

46. A method of imaging a biological system, the method comprising providing an imaging system having an optical axis, placing the biological system on the optical axis, illuminating the biological system with at least partially coherent light from an illumination direction inclined to the optical axis, and imaging the biological system.

47. A method according to claim 46 wherein the biological system is a monolayer or thin layer of cells.

48. A method according to claim 46 wherein the biological system includes optogenetically-modified cardiomyocytes with depolarizing or hyperpolarizing opsins.

49. An imaging and control system for imaging and controlling live biological systems, the system comprising a detector array arranged to detect light from a field of view and output detector signals, a support arranged to support a biological system in the field of view, an illuminating light source arranged to direct light towards the biological system, a processor arranged to receive the detector signals and generate image data; and a stimulating light source arranged to direct light at the biological system thereby to stimulate activity in the biological system.

50. A system according to claim 49 further comprising a display arranged to receive the image data and to display an image based on the image data.

51. A system according to claim 49 wherein the stimulating light source is arranged to generate stimulating light patterns having a plurality of different forms, thereby to stimulate different areas of the biological system.

52. A system according to claim 49 wherein the stimulating light source is further arranged to generate light arranged to suppress activity in the biological system;
wherein the processor is arranged to perform an analysis of the image data to determine one or more parameters of the image data, or one or more parameters of the biological system;
to compare one or more of the parameters with one or more reference values to determine whether the biological system meets one or more criteria, and to generate an output on the basis of that determination; and
wherein the processor is arranged to control stimulating light source and to vary its optical output in response to the analysis of the image data, while an imaging system images the biological system to provide real time control of activity of the biological system.

53. A method of imaging and controlling an optogenetic biological system, the method comprising providing an imaging system, placing the biological system in the field of view of the imaging system, optically stimulating or suppressing activity in the biological system using a first light source, illuminating the biological system using a second light source, and imaging the biological system.

54. A method according to claim 19 wherein the optical stimulation or suppression of activity in the biological system, the illumination of the biological system and the imaging of the biological system are performed simultaneously, andwherein the imaging of the biological system is performed over an imaging period, and the stimulation or suppression is varied during the imaging period to provide real-time control of the activity.
